(19) United States
(12) Patent Application Publication
(10) Pub. No.: US 2014/0074036 A1
(43) Pub. Date: Mar. 13, 2014

(54) MEDICAL STRAPS

(71) Applicant: TAIWAN PAIHO LIMITED, Ho Mei Town (TW)

(72) Inventor: Allen CHENG, Ho Mei Town (TW)

(73) Assignee: TAIWAN PAIHO LIMITED, Ho Mei Town (TW)

(21) Appl. No.: 13/680,765

(22) Filed: Nov. 19, 2012

(30) Foreign Application Priority Data
Sep. 7, 2012 (TW) 101217381

(51) Int. Cl. A61M 25/02

(52) U.S. Cl. CPC A61M 25/02
USPC 604/179

(57) ABSTRACT

Provided is a medical strap having an upper strip and a lower strip. An upper surface of the upper strip is a rough surface, while a lower surface thereof has floss disposed thereon. A plurality of hooks are disposed on an upper surface of the lower strip, and a lower surface of the lower strip is a smooth surface. Transverse and longitudinal parting lines are formed in a region on the upper strip, and therefore movable and securing portions are formed. A medical tubing is secured by the movable and securing portions.

### BACKGROUND OF THE INVENTION

1. Technical Field

The present invention relates to a medical strap, and in particular to a medical strap for securing a medical tubing.

2. Description of the Prior Art

Various tubings are often used in medical field. For medical and nursing techniques including injection, swathing, intubation, etc., tubings are necessarily used in injection and intubation for transmitting or storing liquid. In addition, tubings of aseptic materials are also frequently used for an medical operation, so that the surgery can be smoothly performed without causing any infection. In order to avoid displacements of these tubings due to the movement or the posture change of the patient, these tubings have to be carefully secured to minimize the possibility of falling off after a correct insertion of these tubings. Besides, some patients may pull or move these tubings when being injected and cause the transmission of liquid inside the tubings to fail. As a result, the tubings need to be secured over and over again in one single day to make sure the treatment can be continued. This will increase the loading of nursing staffs. Also, loose medical tubings may be medically troublesome for patients, and even cause other injuries to the patients. This may cause problems to medical safety.

### SUMMARY OF THE INVENTION

In order to solve the problems mentioned above, the main objective of the present invention is to provide a medical strap for securing a medical tubing.

According to the objective above, the present invention provides a medical strap, comprising: a first strip having opposingly arranged first surface and second surface, a plurality of hooks being disposed on the first surface, the second surface being a smooth surface; and a second strip having opposingly arranged third surface and fourth surface, a floss being disposed on the third surface, the fourth surface being a rough surface, wherein the floss on the third surface is hooked by the hooks on the first surface to join the first strip and the second strip together, and wherein a region on the second strip is provided with two first parting lines and a second parting line, the first parting lines are of the same length, parallel to the longitudinal direction of the second strip, spaced apart by a distance, and respectively located on both sides of a central line of symmetry of the second strip, the second parting line is perpendicular to the first parting lines, such that at least one securing portion and at least two movable portions are formed in the region on the second strip, and the movable portions are respectively located on both sides of the second parting line.

Another objective of the present invention is to provide a medical strap employing a combination of a securing portion and a movable portion for securing a medical tubing and reducing the displacement thereof.

According to the objective above, the present invention provides a medical strap, comprising: a first strip having opposingly arranged first surface and second surface, a plurality of hooks being disposed on the first surface, the second surface being a smooth surface; and a second strip having opposingly arranged third surface and fourth surface, a floss being disposed on the third surface, the fourth surface being a rough surface, wherein the floss on the third surface is hooked by the hooks on the first surface to join the first strip and the second strip together, and wherein a region on the second strip is provided with two first parting lines and two second parting lines, the first parting lines are of the same length, parallel to the longitudinal direction of the second strip, spaced apart by a distance, and respectively located on both sides of a central line of symmetry of the second strip, the second parting lines respectively are perpendicular to the first parting lines, and respectively intersect two opposing edges of the second strip, such that one securing portion and four movable portions are formed in the region on the second strip, and the movable portions are respectively located on both sides of the second parting lines.

Still another objective of the present invention is to provide a medical strap which is capable of simultaneously securing a plurality of medical tubings.

According to the objective above, the present invention provides a medical strap, comprising: a first strip having opposingly arranged first surface and second surface, a plurality of hooks being disposed on the first surface, the second surface being a smooth surface; and a second strip having opposingly arranged third surface and fourth surface; a floss being disposed on the third surface, the fourth surface being a rough surface, wherein the floss on the third surface is hooked by the hooks on the first surface to join the first strip and the second strip together, and wherein a region on the second strip is provided with two first parting lines and a second parting line, the first parting lines are of the same length, parallel to the longitudinal direction of the second strip, spaced apart by a distance, and respectively located on both sides of a central line of symmetry of the second strip, the second parting line is perpendicular to the first parting lines, and intersects the first parting lines, such that two securing portions and two movable portions are formed in the region on the second strip, and the movable portions are respectively located on both sides of the second parting line.

The medical trap proposed in the present invention is advantageous in reducing the displacement of the medical tubing during the securing process, and being capable of simultaneously securing a plurality of medical tubings. Adjustment may be made to the securing method according to the diameter of the medical tubing for optimizing the effect of securing the medical tubing.

### BRIEF DESCRIPTION OF THE DRAWINGS

FIG. 1 is a schematic view of a medical strap according to a first embodiment of the present invention.

FIG. 2 is a schematic view of a medical strap according to a second embodiment of the present invention.

FIG. 3 is a schematic assembled view of movable portions on the left half of the medical strap according to the first embodiment of the present invention.

FIG. 4 is a schematic assembled view of movable portions on the left half of the medical strap according to the second embodiment of the present invention.

FIG. 5 is a schematic view showing a process of securing a medical tubing with the medical strap according to the first embodiment of the present invention.

FIG. 6 is a schematic view showing the completed securing of the medical tubing using the medical strap according to the first embodiment of the present invention.

FIG. 7 is a schematic view showing a process of securing a medical tubing with the medical strap according to the second embodiment of the present invention.

FIG. 8 is a schematic view showing the completed securing of the medical tubing using the medical strap according to the second embodiment of the present invention.

### DETAILED DESCRIPTION OF THE PREFERRED EMBODIMENTS

Proposed is a medical strap having an upper strip and a lower strip. The upper and lower strips are bound together by joining a plurality of hooks and floss. The joining of the “hooks” and “floss” is achieved by the currently-available fastening techniques, so it will not be depicted in detail in the following description. In addition, the following drawings are not drawn to their actual dimensions, and serve as schematic views for illustrating the features of the present invention.

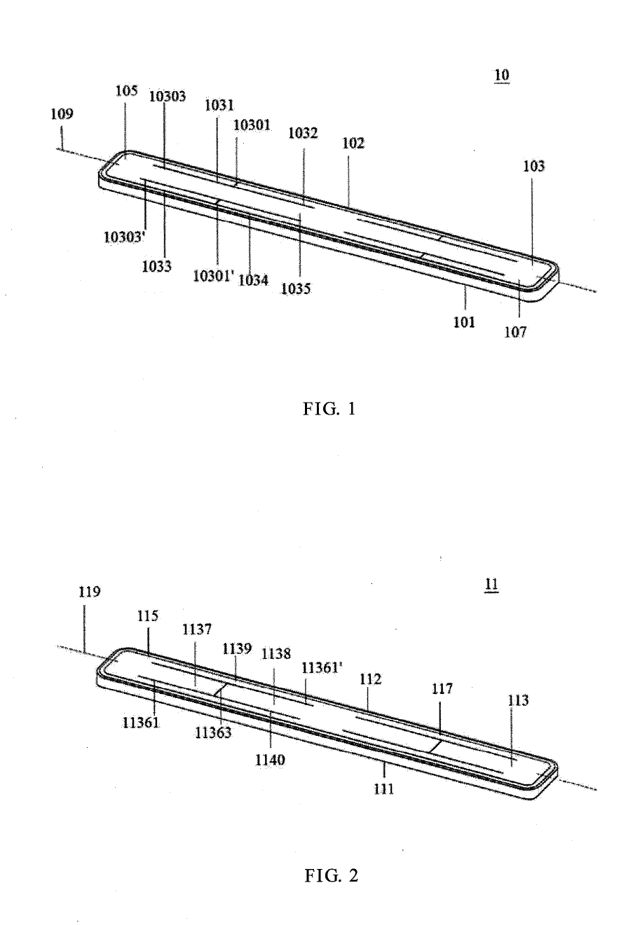

First, please refer to FIG. 1, which is a schematic view of a medical strap according to a first embodiment of the present invention. As shown in FIG. 1, the medical strap 10 of the present invention comprises a lower strip 101 and an upper strip 103. The lower strip 101 is opposingly attached to the upper strip 103. Two transverse parting lines 10303, 10303′ and two longitudinal parting lines 10301, 10301′ are provided on the left half 105 of the upper strip 103. The two transverse parting lines 10303, 10303′ are of the same length and parallel to the longitudinal direction of the upper strip 103. The two transverse parting lines 10303, 10303′ separate from each other by a distance, and are located respectively on both sides of a central line of symmetry 109 in the longitudinal direction of the upper strip 103. The two longitudinal parting lines 10301, 10301′ are respectively perpendicular to the two transverse parting lines 10303, 10303′, and respectively intersect two opposite edges 102 of the upper strip 103, thereby forming a securing portion 1035 and four movable portions 1031, 1032, 1033, 1034. The securing portion 1035 is located between the two transverse parting lines 10303 and 10303′. The movable portion 1031 is defined by the transverse parting line 10303, longitudinal parting line 10301 and an edge 102 of the upper strip 103. Two movable portions 1031 and 1032 are respectively located on both sides of the longitudinal parting line 10301, and two movable portions 1033 and 1034 are respectively located on both sides of the longitudinal parting line 10301′. A right half 107 symmetrical to the left half 105 also has a parting line structure which is similar to that of the left half 105, wherein the locations of the parting lines are similar to those mentioned above, and will not be described again. The lower strip 101 and upper strip 103 of this medical strap 10 are made of polymer materials. It is to be noted that the medical strap 10 is made without using bonding processes such as thermo-compression bonding, ultrasonic bonding, or high frequency bonding.

Next, please refer to FIG. 2, which is a schematic view of a medical strap according to a second embodiment of the present invention. As shown in FIG. 2, a medical strap 11 of this embodiment comprises: a lower strip 111 and an upper strip 113. The lower strip 111 is opposingly attached to the upper strip 113. Two transverse parting lines 11361, 11361′ and one longitudinal parting line 11363 are provided on the left half 115 of the upper strip 113. The two transverse parting lines 11361, 11361′ are of the same length and parallel to the longitudinal direction of the upper strip 113. The two transverse parting lines 11361, 11361′ separate from each other by a distance, and are located respectively on both sides of a central line of symmetry 119 in the longitudinal direction of the upper strip 113. The longitudinal parting line 11363 is perpendicular to and intersects the two transverse parting lines 11361, 11361′, thereby forming two securing portions 1139 and 1140 as well as two movable portions 1137 and 1138. The two securing portions 1139 and 1140 are located respectively on both sides of the longitudinal parting line 11363. The two movable portions are located between the transverse parting lines 11361′ and 11361 and an edge 112 of the upper strip 113, while the movable portions 1137 and 1138 are defined by the transverse parting lines 11361, 11361′ and the longitudinal parting line 11363. A right half 117 symmetrical to the left half 115 also has a parting line structure which is similar to that of the left half 105, wherein the locations of the parting lines are similar to those mentioned above, and will not be described again. The lower strip 111 and upper strip 113 of this medical strap 11 are made of polymer materials. It is to be noted that the medical strap 11 is made without using bonding processes such as thermo-compression bonding, ultrasonic bonding, or high frequency bonding.

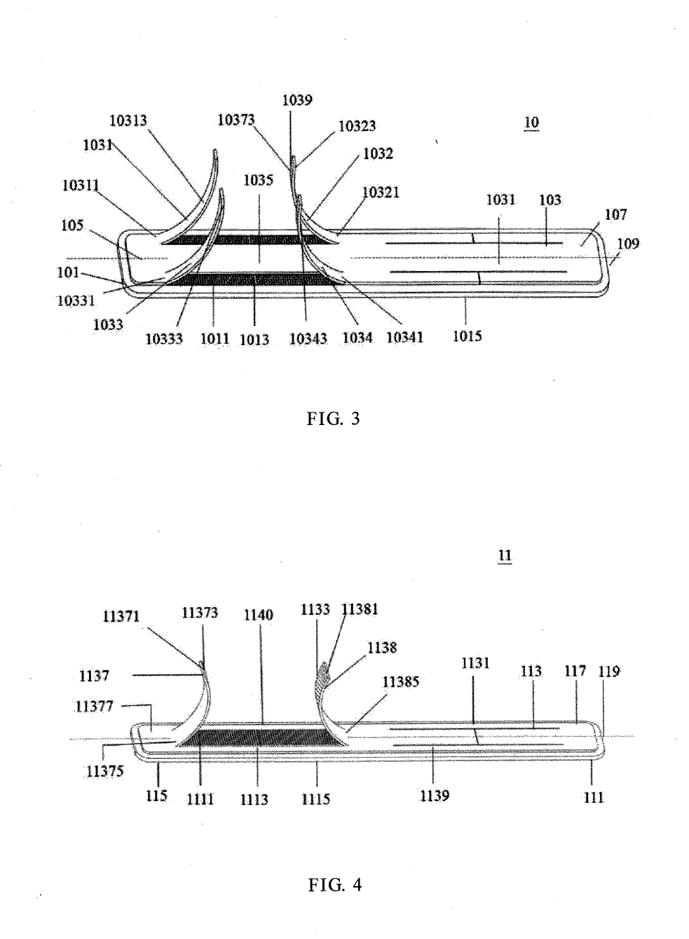

Next, please refer to FIG. 3, which is a schematic view of the movable portions on the left half of the medical strap of the first embodiment of the present invention. As shown in FIG. 3, the lower strip 101 has an upper surface 1013 and a lower surface 1015. The upper surface 1013 backs onto the lower surface 1015. A plurality of hooks 1011 are dispersed on the upper surface 1013. The lower surface 1015 is a smooth surface which may be provided with adhesive, by which the medical strap 10 is secured at a specific position. The upper strip 103 has an upper surface 1031 and a lower surface 1039. The upper surface 1031 backs onto the lower surface 1039. Floss 10373 is dispersed on the lower surface 1039. The floss 10373 on the lower surface 1039 of the upper strip 103 can be hooked by the hooks 1011 on the upper surface 1013 of the lower strip 101, so as to enable the attachment or join between the upper strip 103 and the lower strip 101. As mentioned above with respect to the first embodiment of the present invention, one securing portion 1035 and four movable portions 1031, 1032, 1033, 1034 are formed on the left half 105 of the upper strip 103, the two movable portions 1031 and 1032 are respectively located on both sides of the longitudinal parting line 10301, and the two movable portions 1033 and 1034 are respectively located on both sides of the longitudinal parting line 10301′. One ends of the movable portions 1031, 1032, 1033 and 1034 are fixed ends 10311, 10321, 10331 and 10341, respectively, while the other ends opposite to the fixed ends 10311, 10321, 10331 and 10341 are free ends 10313, 10323, 10333 and 10343, respectively. The free ends 10313 and 10333 of the movable portions 1031 and 1033 are adjacent to the free ends 10323 and 10343 of the movable portions 1032 and 1034, respectively. The movable portions 1031, 1032, 1033 and 1034 have a structure similar to the upper strip 103, which is the same lower surface 1039. On the lower surface 1039 is the floss 10373 which can be hooked by the hooks 1011 on the upper surface 1013 of the lower strip 101 for attachment. In addition, the right half 107 on the side opposite to the left half 105 of the upper strip 103 also has a similar structure.

Next, please refer to FIG. 4, which is a schematic view of the movable portions on the left half of the medical strap according to the second embodiment of the present invention. As shown in FIG. 4, the lower strip 111 has an upper surface 1113 and a lower surface 1115. The upper surface 1113 backs onto the lower surface 1115. A plurality of hooks 1111 are dispersed on the upper surface 1113. The lower surface 1115 is a smooth surface which may be provided with adhesive, by which the medical strap 11 is secured at a specific position. The upper strip 113 has an upper surface 1131 and a lower surface 1133. The upper surface 1131 backs onto the lower surface 1133. Floss 11373 is dispersed on the lower surface 1133. The floss 11373 on the lower surface 1133 of the upper strip 113 can be hooked by the hooks 1111 on the upper surface 1113 of the lower strip 111, so as to enable the attachment between the upper strip 113 and the lower strip 111. As mentioned above with respect to the second embodiment of the present invention, two securing portions 1139, 1140 and two movable portions 1137, 1138 are formed on the left half 115 of the upper strip 113, the two movable portions 1137 and 1138 are respectively located on both sides of the longitudinal parting line 11363 and between the two securing portions 1139 and 1140. One ends of the movable portions 1137 and 1138 are fixed ends 11375 and 11385, respectively, while the other ends opposite to the fixed ends 11375 and 11385 are free ends 11371 and 11381, respectively. The free end 11371 is adjacent to the free end 11381. The movable portions 1137 and 1138 have a structure similar to the upper strip 113, which is the same lower surface 1133. On the lower surface 1133 is the floss 11373 which can be hooked by the hooks 1111 on the upper surface 1113 of the lower strip 111 for attachment. In addition, the right half 117 on the side opposite to the left half 115 of the upper strip 113 also has a similar structure.

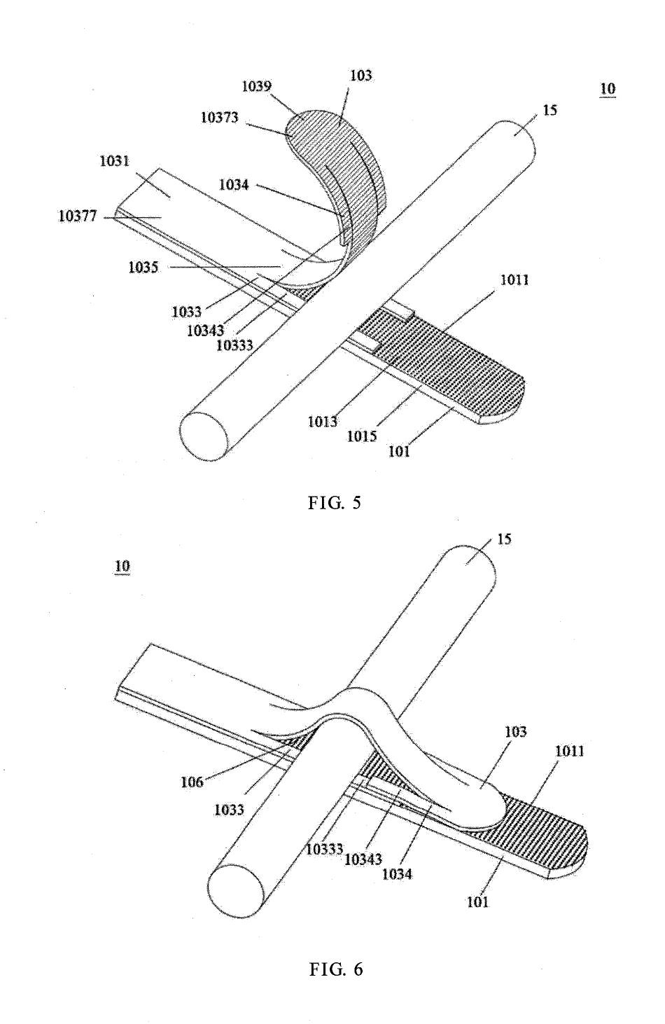

Next, please refer to FIG. 5 and FIG. 6, which are schematic views showing a process of securing a medical tubing with the medical strap according to the first embodiment of the present invention. As shown in FIG. 5, in the case of the movable portions 1033, 1034 and the securing portion 1035, for adjusting the tightness which is provided to the medical tube 15 by the securing portion 1035, adjustment can be made with the movable portions 1033, 1034 and the securing portion 1035 according to the diameter of a medical tubing 15, so that the displacement of the medical tubing 15 can be reduced. Specifically, at first, the upper strip 103 is not fully attached to the lower strip 101, and the movable portion 1034, which has a structure similar to the upper strip 103 and shares the same upper surface 1031, is not attached to the lower strip 101, either. Next, the medical tubing 15 is placed below the securing portion 1035 and above the upper surface 1031 of the movable portion 1033, on which a rough surface 10377 is provided. The rough surface 10377 is used for increasing the friction between the medical tubing 15 and the movable portion 1033, so that the movable portion 1033 can reduce the displacement of the medical tubing 15. Then, the movable portion 1034 is attached to the lower strip 101. Furthermore, since the lower surface 1039 of the securing portion 1035, the movable portion 1034 and the free end 10343 of the movable portion 1034 is the lower surface 1039 of the upper strip 103, the securing portion 1035, the lower surface 1039 of the movable portion 1034 and the free end 10343 of the movable portion 1034 is also provided with the floss 10373.

In the process of securing the medical tubing 15 with the medical strap 10, the floss 10373 provided on the lower surface 1039 of the securing portion 1035 will contact the medical tubing 15, and thereby clinging to the medical tubing 15. Next, the floss 10373 on the lower surface 1039 of the free end 10343 of the movable portion 1034 is hooked by the plurality of hooks 1011 provided on the upper surface 1013 of the lower strip 101 for attachment. Therefore, the free ends 10333, 10343 of the movable portions 1033, 1034 of the upper strip 103 will overlappingly engaged. In the formed structure, the movable portion 1034 is above the movable portion 1033, the securing portion 1035 is arched and thus forms a space 106 together with the lower strip 101. For securing the medical tubing 15, the user presses the securing portion 1035, so the floss 10373 provided on the lower surface 1039 of the securing portion 1035 is hooked by the hooks 1011 on the upper surface 1013 of the lower strip 101 for attachment. In such a way, the space 106 for the medical tubing 15 is gradually decreased, thereby securing the medical tubing 15 and reducing the displacement thereof. The secured medical tubing is shown in FIG. 6. Such method for securing the medical tubing 15 is also applicable for the combination of the other movable portions 1031, 1032 and the securing portion 1035. The aforementioned method for securing the medical tubing 15 is also applicable to the structure of the right half 107 that is a symmetric half of the left half 105. Adjustment may be made to the securing method according to the diameter of the medical tubing 15 for optimizing the effect of securing the medical tubing 15.

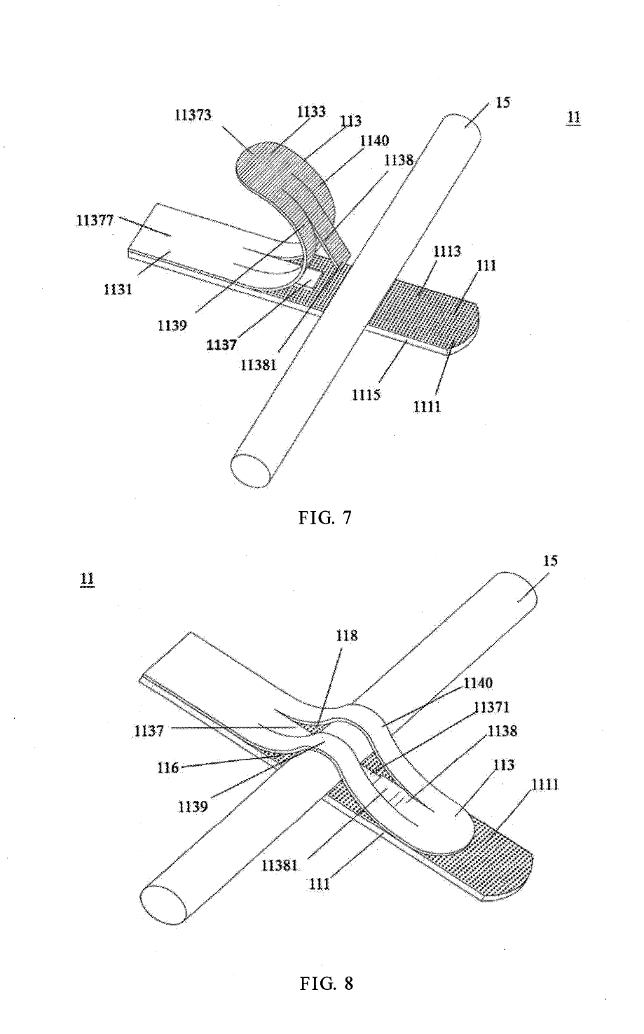

Next, please refer to FIGS. 7 and 8, which are schematic views showing a process of securing a medical tubing with the medical strap according to the second embodiment of the present invention. As shown in FIG. 7, in the case of the movable portions 1137, 1138 and the securing portions 1139 and 1140, for adjusting the tightness provided to the medical tube 15, adjustment can be made with the movable portions 1137, 1138 and the securing portions 1139, 1140 according to the diameter of a medical tubing 15, so that the displacement of the medical tubing 15 can be reduced. At first, the upper strip 113 is not fully attached to the lower strip 111, and the movable portion 1138 is not attached to the lower strip 111, either, wherein the movable portions 1137 and 1138 have a structure similar to the upper strip 113 and share the same upper surface 1131. Next, the medical tubing 15 is placed below the two securing portions 1139, 1140 and above the upper surface 1131 of the movable portion 1137, on which a rough surface 11377 is provided. The rough surface 11377 is used for increasing the friction between the medical tubing 15 and a contacting surface of the medical strap 11, so that the medical strap 11 can reduce the displacement of the medical tubing 15. Furthermore, since the lower surface 1133 of the securing portions 1139 and 1140, the movable portions 1137 and 1138, and the free ends 11371 and 11381 of the movable portions 1137 and 1138 is the lower surface 1133 of the upper strip 113, the securing portions 1139 and 1140, the movable portions 1137 and 1138, and the free ends 11371 and 11381 of the movable portions 1137 and 1138 is also provided with the floss 11373.

In the process of securing the medical tubing 15 with the medical strap 11, the floss 11373 provided on the lower surface 1133 of the securing portions 1139 and 1140 will contact the medical tubing 15, and the floss 11373 on the lower surface 1133 of the free end 11381 of the movable portion 1138 will also contact the medical tubing 15, therefore they are hooked by the plurality of hooks 1111 provided on the upper surface 1113 of the lower strip 111 for attachment. Thus, the free end 11381 of the movable portion 1138 of the upper strip 133 will partially overlappingly engaged with the free end 11371 of the movable portion 1137. In the formed structure, the free end 11381 is above the free end 11371, the securing portions 1139 and 1140 are arched and thus respectively form two spaces 116 and 118 together with the lower strip 111. For securing the medical tubing 15, the user presses the securing portions 1139 and 1140, so the floss 11373 provided on the lower surface 1133 is hooked by the hooks 1111 on the upper surface 1113 of the lower strip 111 for attachment. In such a way, the spaces 116 and 118 for the medical tubing 15 are gradually decreased, thereby securing the medical tubing 15 and reducing the displacement thereof. The secured medical tubing is shown in FIG. 8. The aforementioned method for securing the medical tubing 15 is also applicable to the structure of the right half 117 that is a symmetric half of the left half 115. Adjustment may be made to the securing method according to the diameter of the medical tubing 15 for optimizing the effect of securing the medical tubing 15.

Although the present invention has been disclosed with the preferred embodiments above, those embodiments are not for limiting the present invention. Those skilled in the art may make several alterations and modifications without departing from the spirit and scope of the present invention. The scope of present invention is therefore defined with the appended claims.

What is claimed is:

1. A medical strap, comprising:
a first strip having opposingly arranged first surface and second surface, a plurality of hooks being disposed on the first surface, the second surface being a smooth surface; and
a second strip having opposingly arranged third surface and fourth surface, a floss being disposed on the third surface, the fourth surface being a rough surface,
wherein the floss on the third surface is hooked by the hooks on the first surface to join the first strip and the second strip together, and
wherein a region on the second strip is provided with two first parting lines and a second parting line, the first parting lines are of the same length, parallel to the longitudinal direction of the second strip, spaced apart by a distance, and respectively located on both sides of a central line of symmetry of the second strip, the second parting line is perpendicular to the first parting lines, such that at least one securing portion and at least two movable portions are formed in the region on the second strip, and the movable portions are respectively located on both sides of the second parting line.

2. The medical strap of claim 1, wherein one securing portion and four movable portions are formed in the region on the second strip, the securing portion is located between the first parting lines, and one of the movable portions is defined by one of the first parting lines, the second parting line and an edge of the second strip.

3. The medical strap of claim 1, wherein two securing portions and two movable portions are formed in the region on the second strip, each of the securing portions is located between one of the first parting lines and an edge of the second strip, each of the movable portions is defined by the first parting lines and the second parting line.

4. The medical strap of claim 1, wherein the material of the first strip and the second strip is a polymeric material.

5. A medical strap, comprising:
a first strip having opposingly arranged first surface and second surface, a plurality of hooks being disposed on the first surface, the second surface being a smooth surface; and
a second strip having opposingly arranged third surface and fourth surface, a floss being disposed on the third surface, the fourth surface being a rough surface,
wherein the floss on the third surface is hooked by the hooks on the first surface to join the first strip and the second strip together, and
wherein a region on the second strip is provided with two first parting lines and two second parting lines, the first parting lines are of the same length, parallel to the longitudinal direction of the second strip, spaced apart by a distance, and respectively located on both sides of a central line of symmetry of the second strip, the second parting lines respectively are perpendicular to the first parting lines, and respectively intersect two opposing edges of the second strip, such that one securing portion and four movable portions are formed in the region on the second strip, and the movable portions are respectively located on both sides of the second parting lines.

6. A medical strap, comprising:
a first strip having opposingly arranged first surface and second surface, a plurality of hooks being disposed on the first surface, the second surface being a smooth surface; and
a second strip having opposingly arranged third surface and fourth surface, a floss being disposed on the third surface, the fourth surface being a rough surface,
wherein the floss on the third surface is hooked by the hooks on the first surface to join the first strip and the second strip together, and
wherein a region on the second strip is provided with two first parting lines and a second parting line, the first parting lines are of the same length, parallel to the longitudinal direction of the second strip, spaced apart by a distance, and respectively located on both sides of a central line of symmetry of the second strip, the second parting line is perpendicular to the first parting lines, and intersects the first parting lines, such that two securing portions and two movable portions are formed in the region on the second strip, and the movable portions are respectively located on both sides of the second parting line.
